(19) United States
(12) Patent Application Publication
(10) Pub. No.: US 2003/0190313 A1
(43) Pub. Date: Oct. 9, 2003

(54) Diagnostic tests for anti-endotoxin core antibodies

(76) Inventors: Daniel P. Rossignol, Mahwah, NJ (US); Melvyn Lynn, East Brunswick, NJ (US)

Correspondence Address: CLARK & ELBING LLP, 101 FEDERAL STREET, BOSTON, MA, 02110, US

(21) Appl. No.: 10/344,183

(22) PCT Filed: Aug. 7, 2001
(86) PCT No.: PCT/US01/41627

(51) Int. Cl. G01N 33/53; G01N 33/537; G01N 33/543; A61K 39/395

(52) U.S. Cl. 424/130.1; 435/7.92

(57) ABSTRACT

The invention provides methods and kits for determining whether a patient may benefit from treatment with an anti-endotoxin compound.

### BACKGROUND OF THE INVENTION

This invention relates to diagnostic tests for detecting anti-endotoxin core antibodies.

The incidence of gram-negative bacteremia in the United States has been estimated to be approximately 100,000 to 300,000 cases per year, with a mortality rate of 30-60%. Endotoxin, which is a lipopolysaccharide (LPS) component of the cell wall of gram-negative bacteria and induces an “innate” immune response, is shed from the membranes of growing and dying bacteria. In most cases, during bacterial infection, this innate immune response warns the body that a bacterial infection is present, causing the immune system to mount an antimicrobial attack. However, an overwhelming immune response to endotoxin can be pathological, leading to Systemic Inflammatory Response Syndrome (SIRS) and, possibly, shock. The symptoms of these conditions include fever, generalized inflammation, disseminated intravascular coagulation (DIC), hypotension, acute renal failure, acute respiratory distress syndrome (ARDS), hepatocellular destruction, and cardiac failure.

Endotoxin has been suggested to be a causative agent of a large number of complications from surgery. However, while it has been clearly established that blood or plasma endotoxin can be detected during and after surgery, it is often found without a locus of bacterial infection (i.e., an endotoxin source) (Andersen et al., J. Thorac. Cardiovasc. Surg. 93(1):115-119, 1987). It is now believed possible that this endotoxin may come from the gut (Martinez-Pellus et al., Intensive Care Med. 23(12):1251-1257, 1997). Apparently, the ability of the mucosal barrier of the intestine to block translocation of endotoxin from inside the intestine to the blood supply may be compromised by gut ischemia due to hypoperfusion, which occurs when blood circulation and oxygenation of the intestines is impaired (Oudemans-van Straaten et al., J. Cardiothorac. Vasc. Anesth. 10(2):187-194, 1996). Such hypoperfusion can occur during cardiac failure (Niebauer et al., Lancet 353(9167):1838-1842, 1999) or during coronary bypass graft surgery (Martinez-Pellus et al., supra).

Recently, antibodies have been described that cross-react with a wide variety of endotoxins, from different bacteria. While most antibodies are species-specific (or even strain-specific) for endotoxin, the recently described antibodies react with the endotoxin core, which is common to endotoxins from a wide variety of gram-negative bacteria. This discovery of so-called anti-endotoxin core antibodies (also referred to as EndoCAb) has enabled the detection of a relationship between levels of anti-endotoxin core antibody and outcome from cardiac bypass surgery. In particular, when antibody levels to endotoxin are high prior to surgery, it can be predicted that a patient may be able to neutralize or clear endotoxin during and after surgery. Prior to elective surgery, such as cardiac bypass surgery, candidates with high anti-endotoxin core antibody titers, thus, may be predicted to have a greater chance of uncomplicated surgical outcome (Barclay, Prog. Clin. Biol. Res. 392:263-272, 1995; Bennett-Guerrero et al., J. American Medical Assoc. 277(8):646-650, 1997; Hamilton-Davies et al., Chest 112(5):1189-1196, 1997). Some evidence has been found that anti-endotoxin core antibody levels are also important in the association of complications with non-cardiac elective surgery (Mythen et al., Blood Coagul. Fibrinolysis 4(6):999-1005, 1993).

### SUMMARY OF THE INVENTION

The invention provides methods of determining whether a patient (e.g., a surgical patient) could benefit from treatment with an anti-endotoxin compound. The methods involve obtaining a sample (e.g., a blood, plasma, serum, saliva, or urine sample) from a patient, and detecting the level of anti-endotoxin core antibodies (e.g., IgM or IgG antibodies) in the sample. Detection of a level that is inadequate (e.g., below 250, 100, 90, 80, 70, 60, 50, 40, 30, or 20 MU/ml) may indicate that it is advisable to supplement the poor ability of the patient to neutralize endotoxin through the administration of an anti-endotoxin compound to the patient. The methods of the invention can involve the use of an immunoassay. Preferably, the immunoassay is carried out by the use of a bedside kit, which requires minimal, if any, involvement of a medical laboratory. For example, as is described further below, a strip containing an endotoxin from a gram-negative bacterium (or, preferably, more than one gram-negative bacterium) can be contacted with a patient sample, and anti-endotoxin core antibodies that bind to the strip can be detected. Also as is described further below, an enzyme-linked immunosorbent assay can also be used in the invention. The invention also includes kits that can be used to carry out the methods described herein, as well as methods of treating patients that have or at risk of developing a condition that is treatable by the use of an anti-endotoxin compound. These methods involve (i) determining the level of anti-endotoxin core antibodies in a sample from the patient, and, if the levels of anti-endotoxin core antibodies in the sample are inadequate, (ii) administering an antiendotoxin compound to the patient. The invention also includes the use of anti-endotoxin compounds in such methods, as well as the use of anti-endotoxin compounds in the preparation of medicaments for use in such methods.

The methods and kits of the invention facilitate rapid and convenient determination of whether it may be beneficial to administer an anti-endotoxin compound to a patient, so that effects of complications related to endotoxemia can be treated or prevented in the patient.

Other features and advantages of the invention will be apparent from the following detailed description and the claims.

### DETAILED DESCRIPTION

The invention provides diagnostic methods and kits for use in determining whether a patient, e.g., a surgical patient, could benefit from treatment with an anti-endotoxin compound. In particular, the methods and kits of the invention are used to detect the level of anti-endotoxin core antibodies (EndoCAb) in a patient sample. It is advantageous to detect these antibodies because, as is discussed above, while most endotoxin antibodies are species-specific (or even strain-specific), anti-endotoxin core antibodies react with the endotoxin core, which is common to endotoxins from a wide variety of gram-negative bacteria. Antibodies to endotoxin core, in cross-reacting with a wide variety of endotoxins, from different bacteria, are beneficial in neutralizing the toxicity of endotoxin from a wide variety of bacteria. Quantitative measurement of such antibodies has very wide applicability.

According to the invention, detection of low levels of anti-endotoxin core antibodies in a patient sample indicates that the patient may be susceptible to complications of infection by gram-negative bacteria. It may be advisable, thus, to administer an anti-endotoxin compound to such a patient, particularly if the patient is going to be put at risk of endotoxin exposure. In contrast, if the levels of anti-endotoxin core antibodies in a patient sample are high, it may be determined that such treatment is not necessary. As an example, as is noted above, endotoxemia is a risk associated with surgery. Use of the present invention facilitates a determination of whether it may be beneficial to treat a patient with an anti-endotoxin compound before, during, or after surgery. If antibody levels to endotoxin are low prior to surgery, it may be advisable to administer such a compound to the patient, while if the levels are high, such treatment may not be necessary. Also, if anti-endotoxin core antibody levels are found to decrease during or after surgery, it may then be advisable to administer an anti-endotoxin compound.

The methods of the invention can employ any of a number of formats. Preferable formats are those that are amenable to “bedside” use, which enable a medical professional to determine whether a patient has low levels of anti-endotoxin core antibodies, rapidly, conveniently, and inexpensively, with minimal, if any, use of a medical laboratory. However, formats that require the use of a medical laboratory are also included in the invention. Such rapid and inexpensive determination is useful for determining the necessity of prophylactic or therapeutic anti-endotoxin therapy.

In one example of these methods, an adherent medium, such as a plastic strip, containing surface bound endotoxin (preferably a mixture of endotoxins; see below) is contacted with an appropriate dilution of a patient sample (e.g., blood, plasma, serum, urine, or saliva), and any anti-endotoxin core antibodies present in the sample are detected by binding to the medium. The sample is diluted so that an amount of anti-endotoxin core antibodies (e.g., IgM, IgG, or IgA) in the sample that is below the level at which a patient can be said to not be at risk of an endotoxin-related complication (e.g., a level below 250 MU/ml, e.g., a level below 100, 90, 80, 70, 60, 50, 40, 30, or 20 MU/ml) is not detectable on the medium, while an amount of antibody that is at or above the level at which a patient is said to not be at such risk is detectable. The amount of dilution can be, for example, in the case of blood, about 1:100 to 1:500, e.g., 1:200. After incubation with the sample, the strip is washed, and then contacted with a labeled (e.g., enzyme-labeled (e.g., alkaline phosphatase-labeled)) anti-human immunoglobulin antibody (e.g., an anti-IgM, IgG, or IgA antibody), the strip is washed again, a substrate for the enzyme is added, and the level of antibody bound to the medium is detected by measurement of a reaction product of the enzyme and substrate.

Another example of an assay format that can be used in the invention involves the use of a chromatography medium, such as a paper strip, that contains surface bound endotoxin, or, preferably a mixture of endotoxins (see below), at or near one end. This end of the strip is contacted with a diluted sample from a patient, and it is determined whether a complex forms between the endotoxin and any antibodies in the sample by chromatography. If a sufficient level of anti-endotoxin core antibodies is detected in the sample, the patient is in a low risk group for endotoxin-related complications, and treatment with an anti-endotoxin compound may not be needed. However, if such antibodies are not detected in the patient sample, but are detected in a control sample, run in parallel, the patient may be at risk of endotoxin-related complications, and treatment with an anti-endotoxin compound may be advisable.

Yet another example of a test format that can be used in the invention is an enzyme-linked immunosorbent assay (ELISA), which can be used to detect anti-endotoxin core antibodies levels in a patient sample, such as whole blood or serum (Bennett-Guerrero et al., JAMA 277(8):646-650, 1997; Hamilton-Davies et al., Chest 112(5):1189-1196, 1997; Barclay, “Endogenous Endotoxin-Core Antibody (EndoCAb) as a Marker of Endotoxin Exposure: A Review,” In Levin et al., eds., “Bacterial Endotoxins: Lipopolysaccharides From Genes to Therapy,” New York, N.Y., Wiley-Liss, 263-272, 1995; Barclay et al., Infect. Immun. 55:2706-2714, 1987; DiaPharma EndoCAb kit, DiaPharma Group, Inc., West Chester, Ohio). In such an assay, for example, equimolar amounts of incomplete-core rough mutant lipopolysaccharide from each of four species of gram-negative bacteria (Escherichia coli, Pseudomonas aeruginosa, Klebsiella aerogenes, and Salmonella typhimurium) are complexed with polymyxin B, mixed in a cocktail in carbonate-bicarbonate buffer (pH 9.6), and the cocktail is coated on 96-well polystyrene microplates. IgG and IgM anti-endotoxin core antibody concentrations are determined with alkaline phosphatase-conjugated antibodies specific for human gammaglobulin (Zymed Laboratories, Cambridge BioScience, Cambridge, United Kingdom). Test sera, which can be diluted, for example, 1:200, are compared in an ELISA to a reference serum calibrated in anti-endotoxin core antibody median units (MU), where 100 is the median value for IgG or IgM of 1000 healthy adults that are, for example, in a particular locality. Serum can be tested for total IgG and IgM concentrations by laser nephelometry (Whicher et al., Ann. Clin. Biochem. 15:77-85,1978).

The invention also includes kits that can be used to conduct any of the methods described above. Such kits can include, for example, strips of adherent medium to which endotoxin is bound, chromatography strips including endotoxin, or polystyrene plates with endotoxin bound to the plates. Preferably, the kits also include a control anti-endotoxin core antibody sample, having levels of anti-endotoxin core antibodies that are similar to those in an unaffected individual. The kits also can include instructions that state what levels of anti-endotoxin core antibodies (e.g., a level below 250, 100, 90, 80, 70, 60, 50, 40, 30, or 20 MU/ml) indicate that a patient could benefit from treatment with an anti-endotoxin compound.

Anti-endotoxin compounds that can be used to treat a patient identified as in need of such treatment, according to the invention, include, for example, Compound 1287 (SGEA) (U.S. Pat. No. 5,935,938) and Compound B531 (U.S. Pat. No. 5,530,113), as well as other compounds described in these patents and the following U.S. patents: U.S. Pat. No. 5,612,476, U.S. Pat. No. 5,756,718, U.S. Pat. No. 5,843,918, U.S. Pat. No. 5,750664, and U.S. Pat. No. 5,681,824. For example, the anti-endotoxin compound can have the formula:

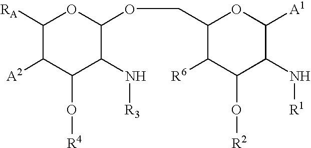

where R¹ is selected from the group consisting of:

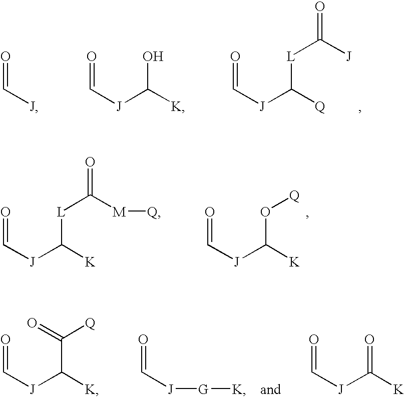

where each J, K, and Q, independently, is straight or branched C1 to C15 alkyl; L is O, NH, or CH₂; M is O or NH; and G is NH, O, S, SO, or SO₂, R² is straight or branched C5 to C15 alkyl; R³ is selected from the group consisting of straight or branched C5 to C18 alkyl,

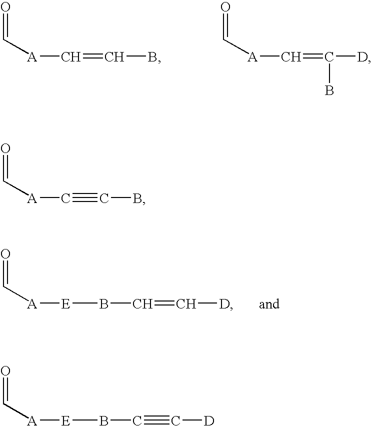

where E is NH, O, S, SO, or SO₂; each A, B, and D, independently, is straight or branched C1 to C15 alkyl; R⁴ is selected from the group consisting of straight or branched C4 to C20 alkyl, and

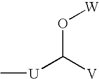

where each U and V, independently, is straight or branched C2 to C15 alkyl and W is hydrogen or straight or branched C1 to C5 allyl;

R_{A} is R⁵ or R⁵—O—CH₂—, R⁵ being selected from the group consisting of hydrogen, J′, —J′—OH, —J′—O—K′, —J′—O—K′—OH, and —J′—O—PO(OH)₂, where each J′ and K′, independently, is straight or branched C1 to C5 alkyl;

R⁶ is selected from the group consisting of hydroxy, halogen, C1 to C5 alkoxy, and C1 to C5 acyloxy; A¹ and A², independently, are selected from the group consisting of

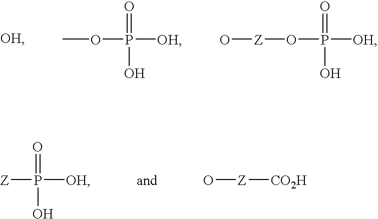

where Z is straight or branched C1 to C10 alkyl; or pharmaceutically acceptable salts thereof.

A preferred, specific example of a compound that can be used in the invention, compound 1287 (SGEA), has the following structure:

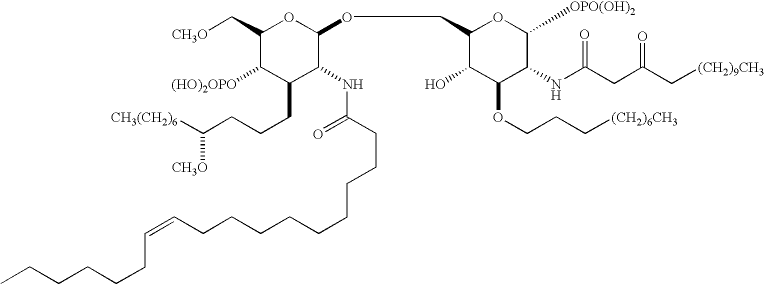

The methods of the invention can be used in conjunction with any type of surgery or medical procedure that could lead to the occurrence of endotoxemia or sepsis syndrome, for example, cardiac surgery (e.g., cardiac bypass, cardiopulmonary bypass, or valve replacement), abdominal surgery, transplantation (of, e.g., liver, heart, kidney, or bone marrow), cancer surgery (e.g., removal of a tumor), major orthopedic surgery (e.g., revision hip arthroplasty or fusion/instrumentation of multiple lumbar or thoracic vertebrae), major general surgery (e.g., any laparotomy expected to exceed 2 hours duration, including partial hepatectomy, pancreatic surgery, or colon surgery), major urological surgery (e.g., radical cystectomy or radical nephrectomy), major vascular surgery (e.g., abdominal aortic aneurysm repair), and major gynecological surgery (e.g., cancer debulking procedure or abdominal hysterectomy with oophorectomy). Additional examples of surgical procedures with which the methods of the invention can be used are surgery for treating acute pancreatitus or inflammatory bowel disease, placement of a transjugular intrahepatic portosystemic stent shunt, hepatic resection, burn wound revision, and burn wound escharectomy.

The methods of the invention can also be used in conjunction with non-surgical procedures in which the gastrointestinal tract is compromised. For example, the methods of the invention can be used in association with chemotherapy or radiation therapy in the treatment of cancer.

The methods of the invention can also be used to determine whether it may be advisable to administer an anti-endotoxin compound to a subject who has or is at risk of developing pulmonary bacterial infection or symptomatic pulmonary exposure to endotoxins and related disorders and conditions. Examples of such disorders and conditions include, for example, cystic fibrosis; immune deficiencies, including immunocompromise due to anti-cancer therapy and immunocompromise due to anti-rejection therapy after organ transplant; asplenia; hypogammaglobulinemia; dysglobulinemias; deficiencies of complement cascade components; HIV or other viral infections; polymorphonuclear granulocyte defects; ciliary dyskinesias (e.g., Kartagener s syndrome); obstructive pulmonary disorders, including congestive heart failure with pulmonary edema, chronic obstructive pulmonary disease, tumors leading to bronchial obstruction, and bronchiectasis (e.g., as a complication of asthma); acute lung injuries that predispose to infection, increase sensitivity to endotoxin, or affect ability to clear endotoxin (e.g., smoke inhalation or heat exposure (e.g., thermal injury, such as by inhalation of hot air or steam)); aspiration of gastric contents; near-drowning; and inhalation of noxious substances.

Anti-endotoxin compounds can be administered to patients identified as being in need of such treatment, according to the methods of the invention, using routes (e.g., injection, infusion, or inhalation) and dosages determined to be appropriate by those of skill in this art. For example, the drug can be administered intravenously for 1-6 hours preoperatively, and administration can be continued for up to 72 hours (e.g., 24 hours) postoperatively. The dose can be, for example, 2-10,000 μg/hour, e.g., 25-3000 μg/hour, or, preferably, 50-1000 μg/hour. Alternatively, the drug can be administered only preoperatively, operatively, postoperatively, or in any combination thereof. The drug is typically administered in a pharmaceutically acceptable formulation, e.g., dissolved in physiological saline, which may include 5% glucose.

In the case of a patient that has or is at risk of developing pulmonary bacterial infection, symptomatic pulmonary exposure to endotoxin, or a related disorder, the administration can be by inhalation, e.g., by the use of an aerosol or a nebulizer. Such administration can be effected by means of periodic bolus administration, by continuous, metered inhalation, or by a combination of the two. A single dose is administered by inhalation of 1 μg-24 mg, for example, 5-150 μg, or, preferably, 10-100 μg of the drug. Of course, recalcitrant disease may require administration of relatively high doses, e.g., 5 mg, the appropriate amounts of which can be determined by one of skill in this art. Appropriate frequency of administration can also be determined by one of skill in this art, and can be, for example, 1-4, for example, 2-3, times each day. Preferably, the drug is administered once each day. In general, treatment is carried out until the symptoms of pulmonary bacterial infection or pulmonary exposure to endotoxin in the patient have lessened to a satisfactory extent or, preferably, have disappeared. It may be necessary, in some cases, to continue administration for several days, e.g., one, two, three, or four weeks.

It will be understood, however, that the specific dose level for any particular patient will depend on a variety of factors, including the activity of the specific compound employed; the age, body weight, general health, and sex of the individual being treated; the time and route of administration; the rate of excretion; other drugs that have previously been administered; and the severity of the particular disease undergoing therapy. Preferably, the administration is carried out prior to the condition that could result in endotoxemia. However, the administration can also be conducted during or after treatment.

All publications cited above are hereby incorporated by reference.

What is claimed is:

1. A method of determining whether a patient could benefit from treatment with an anti-endotoxin compound, said method comprising obtaining a sample from a patient and determining the level of anti-endotoxin core antibodies in the sample.

2. The method of claim 1, wherein the patient sample is selected from the group consisting of blood, plasma, serum, saliva, and urine.

3. The method of claim 1, wherein the anti-endotoxin core antibodies are IgM or IgG antibodies.

4. The method of claim 1, wherein detection of a level of antibodies that is inadequate indicates that the patient could benefit from treatment with an anti-endotoxin compound.

5. The method of claim 1, wherein detection of a level of anti-endotoxin core antibodies of less than 250, 100, 80, 60, 40, or 20 MU/ml indicates that the patient could benefit from treatment with an anti-endotoxin compound.

6. The method of claim 1, wherein the patient has been exposed to, or is at risk of exposure to, endotoxin.

7. The method of claim 6, wherein the patient is a surgical patient.

8. The method of claim 1, wherein determination of the levels of anti-endotoxin core antibodies is conducted using an immunoassay.

9. The method of claim 8, wherein the immunoassay is conducted by use of a bedside kit.

10. The method of claim 9, wherein the kit comprises a strip comprising an endotoxin from a gram-negative bacterium, and said method comprises contacting the strip with the sample and detecting anti-endotoxin core antibodies that bind to the strip.

11. The method of claim 8, wherein the immunoassay is an enzyme-linked immunosorbent assay.

12. A kit for use in determining whether a patient could benefit from treatment with an anti-endotoxin compound, said kit comprising (i) a solid substrate comprising an endotoxin from a gram-negative bacterium, and (ii) instructions stating what level of anti-endotoxin core antibodies detected by use of the kit indicates that the patient could benefit from treatment with an anti-endotoxin compound.

13. A method of treating a patient that has or is at risk of developing a condition that is treatable by the use of an anti-endotoxin compound, said method comprising the steps of (i) determining the level of anti-endotoxin core antibodies in a sample from the patient, and, if the levels of anti-endotoxin core antibodies in the sample are inadequate, (ii) administering an antiendotoxin compound to the patient.
